(19) United States
(12) Patent Application Publication
(10) Pub. No.: US 2007/0122362 A1
(43) Pub. Date: May 31, 2007

(54) HYDROGEL SHEETS AND SHAPES FOR ORAL CARE

(76) Inventors: Martin S. Giniger, New York, NY (US); Matthew S. Spaid, New York, NY (US)

Correspondence Address: BERENBAUM, WEINSHIENK & EASON, P.C, 370 17TH STREET, SUITE 4800, DENVER, CO, 80202, US

(21) Appl. No.: 11/563,634

(22) Filed: Nov. 27, 2006

Related U.S. Application Data
(60) Provisional application No. 60/739,633, filed on Nov. 26, 2005.

(51) Int. Cl. A61K 9/70; A61K 8/19

(52) U.S. Cl. 424/53; 424/443

(57) ABSTRACT

A hydrogel for use in oral care. The hydrogel is ion beam cross-linked, the hydrogel is adapted to be disposed in the oral cavity and may be adapted to provide a denture fixative or may be loaded with a whitening agent for use in whitening one or more teeth and disposed on or adjacent one or more teeth and the whitening agent is slow-released to whiten the one or more teeth. Other loading materials may include materials for treatment of alveolitis or malodor, inter alia. The present invention is a new hydrophilic oral and dental cohesive hydrogel sheet designed to securely grip and cushion prosthetic devices in the human mouth with the further ability to slow release antimicrobial or other orally desirable agents. Additionally, the invention also provides a method of making an orally cohesive device that: 1) is easily adapted and applied to a removable dental prosthesis; 2) bonds well to alveolar ridge/palatal mucosa and denture acrylic materials; and 3) releases cleanly, with no tacky or thixotropic residue when the prosthesis is removed. The cohesive hydrogel gel device is a hydrogel-forming polymer mixed with water, optionally surrounding an internal scrim, and uses an electron-beam energy source to cause cross-linking. The method does not need any chemical additive to affect the cross-linking. Furthermore the beam energy can be adjusted to optimize the cohesive properties of either side of the device, as well as to compensate for addition of orally active agents, if any are chosen. The hydrogel sheets are pre-cut to fit most sizes of maxillary and mandibular full denture prostheses, but can be easily trimmed with a scissors by the end user for the ideal custom fit of any full or partial denture, in either arch.

### CROSS REFERENCE TO RELATED APPLICATIONS

The present invention claims the benefit of and priority from the prior-filed U.S. Provisional Patent Application, No. 60/739,633; filed Nov. 26, 2005, entitled “Hydrogel Sheets and Shapes”; the subject matter of which hereby being specifically incorporated herein by reference for all that it discloses and teaches.

### BACKGROUND

1. Field of Invention

The developments hereof relate to hydrogels in general, and particularly as these may be used in oral care, as in denture fixative technology, for use in treating alveolitis, and/or for controlled release of particular desired substances such as bioactive materials. Of particular use here are electron beam cross-linked hydrogel materials in sheets and/or other shapes.

2. The Prior Art

As a background on hydrogels, generally, it may first be noted that high-water-content hydrogel sheets, cross-linked chemically or by electron beam, have been developed for a variety of uses. Asserted uses in the art have included: skin wound care; medical diagnostics; transdermal drug delivery; cosmetics; skin electrodes; burn healing and burn management; skin cooling; skin moisturizing; skin warming; aroma release or delivery; decongestant release or delivery; adhesive use in skin devices; incontinence devices; and/or vaginal devices. However, a variety of oral, mucosal or dental uses have not apparently been developed.

Previously published hydrogels have typically included: water (about 95% to about 99.5%); a polymer (about 0.5% to about 5%) such as one or more of polyacrylamide, polyethylene glycol, polyvinylpyrrolidone, carboxymethyl cellulose, and/or propylene oxide/ethylene oxide; and a block copolymner. In previous hydrogel alternatives, possible additives have included antimicrobial agents (e.g. AgNO3) and/or pharmaceutically active agents. These would typically have been delivered either passively (transdermally) or actively (iontophoretically) through the skin. In manufacture, the gels maybe cross-linked chemically or using a high-energy electron beam. Electron beam cross-linking of hydrogel materials provides a stable, cohesive form for hydrogel sheets. There are few machines capable of creating such hydrogels. Such a machine is available from Hydrogel Design Systems, Inc., in Langhorne, Pa., USA, a subsidiary of Nesco Industries Inc.

Conventionally, the internal scrim may either be none, or may be a woven or non-woven cotton or plastic fabric or netting embedded within. The thickness may be a uniform thickness of about two hundredths of an inch (0.02″) to about five tenths of an inch (0.50″). The shape and/or dimensions of previous hydrogels have included square, rectangular, round or oval and up to 13″ wide. In a typical wound dressing usage, a 4″×4″ square has been used. For external liners, either plastic, siliconised plastic, paper, or coated paper liners have been used. No external coatings have been taught. For packaging, either Mylar, Plastic, Polyethylene or Siliconized Plastic have been used. Airtight, crimped or heat sealed pouches have also been used to prevent loss of moisture.

As a background on denture fixatives, it may first be noted that various denture fixative agents are known that are sticky or tacky and may swell in contact with water or saliva, thus forming gel-like masses. These masses fill the space between the undersurfaces of the denture plate and the mouth tissue to effect a suction coupling. Known agents have been provided in the form of films, powders and pastes which are placed on the wettened undersurfaces of the dental prosthetic plates. Certain polymers of ethylene oxide are reputed to have fixative properties as shown in U.S. Pat. Nos. 4,280,936 and 4,373,036, inter alia.

Such conventional fixative agents, however, have inherent disadvantages. Uniformity in distribution upon the denture can be important to successful fixation, and such uniformity depends on the care in which the agents are applied by the user to the underside of the denture plate. Moreover, due to saliva, such agents frequently dilute, rapidly resulting in insufficient viscosity to form a good seat and thereby limiting their effectiveness to a short duration. The manufacture of such agents is relatively expensive in that additives must be mixed with the basic agent to improve its flow properties, viscosity and tackiness. Finally, the most unppealing property is that once the prosthesis is removed from the mouth, a residue is left behind on both the denture and the oral tissues that is sticky, messy, bad-tasting and difficult to remove.

Alternatively, there is known a dental adhesive in which there is a compressed fiber mat containing sodium alginate, a dry adhesive, which swells under the action of moisture in the mouth, as described in U.S. Pat. No. 3,990,149. That patent describes the manufacture of a dental adhesive in which sodium alginate is deposited as a dry powder on a non-woven web having thermoplastic fibers. Water is then added so as to produce a semi-hydrated state which causes the second web of non-woven web material to temporarily adhere to the other web by the resulting sticky wetted alginate. This material is then dried by passing it between heated rollers, which also causes the thermoplastic fibers of the non-woven mats to be permanently bonded to each other and thus to become a unitary piece. The products of the prior art adhesive manufacturing methods described have the disadvantage of being non-uniformly bonded and often short-lived in their adhesiveness. Moreover, the systems of the 3,990,149 patent require very expensive and careful quality controls which often result in undetected products of poor quality. Moreover, the aforesaid method requires the use of expensive and time consuming drying ovens. Further, the product deleteriously releases loose fibers in the mouth of the user.

A further improvement of that invention is described in U.S. Pat. No. 4,503,116 that provides a resilient adhesive device of thermally laminated fabrics optionally having a water activated adhesive uniformly dispersed therebetween. Said product was claimed to be suitable for use in the human mouth between a denture and the soft gum tissues. That invention includes a method for manufacturing said device which may more economically produce a high quality product and which avoids the extremely careful controls required in prior art methods. The major disadvantage of this product is that its gripping ability is the lowest of all previous methods. It does not swell to comfortable gel-like mass and the resulting gaps cause dentures to remain loose-fitting. Also this improvement does not describe any method of adding an antimicrobial or other desirable orally active agent that can be slowly released for therapeutic or cosmetic purposes. Finally, although an attempt is made to overcome the low native adhesive properties of said product through adding an external layer of adhesive onto the laminated liner, this again causes the end user to have to remove an undesirable, foul-tasting and uncomfortable tenacious residue from their mouth and denture.

Gapping and gripping issues also plague alveolitis or “dry-socket” treatments. In particular, it is desirable to form tight, no-gap fillings for the holes left after tooth extraction to alleviate the possibility for air or other materials to come into contact with exposed bony or nerve tissue. Otherwise, a highly painful state will ensue. It would also be desirable for at least a controlled, though substantially minute amount of air or oxygen to breathe into the hole(s) for promotion of healing. A controlled release of a pain management substance could be useful as well.

As to other situations for controlled release of bioactive materials, a variety of methods have been developed for providing bioactive material delivery to a human body. However, simplicity and control in oral use is not a characteristic of these methods. Similarly, in tooth whitening, a variety of means and compositions have been used to effectuate or simulate whitening of teeth. The use of hydrogen peroxide or like peroxides are perhaps the most significant. However, controlled delivery of whitening agents remains an issue.

### SUMMARY OF INVENTION

The present invention is directed generally to new hydrophilic oral and dental cohesive hydrogel products that are adapted for a variety of purposes; e.g., to slow- released, slowly-swallowed, smoking cessation and appetite control integrated system; over the counter (i.e., “OTC”) high concentration tooth whitening bio-adhesives dots allowing for superior containment and activated continuous release of a peroxygen bleaching agent; and/or to securely grip and cushion a prosthetic device in a human mouth. In some embodiments, such a product may be provided with a further ability to slowly release antimicrobial or other orally desirable bioactive agents.

Additionally or alternatively, other embodiments may also provide for a method of making an orally cohesive device that: 1) may easily adapt to and be simply applied to a removable dental prosthesis; 2) may bond well to alveolar ridge/palatal mucosa and denture acrylic materials; and 3) releases cleanly, with no tacky or thixotropic residue when the prosthesis is removed. A cohesive hydrogel gel device useful herefor may be a hydrogel-forming polymer mixed with water, optionally surrounding an internal scrim, and using an electron-beam energy source to cause cross-linking. Such a cross-linking method does not need any chemical additive to effect the cross-linking. Furthermore, the beam energy can be adjusted to optimize the cohesive properties of either side of the device, as well as to compensate for addition of orally active agents, if any are chosen. The hydrogel sheets may be pre-cut to fit most sizes of maxillary and mandibular full denture prostheses, but can be easily trimmed with a scissors by the end user for the ideal custom fit of any full or partial denture, in either arch.

Other implementations include a hydrogel cohesive gel sheet that is created for the purpose of holding in place a prosthetic device in the human mouth and a method of producing same, which in some instances further has an ability to slowly release antimicrobial or other orally desirable agents. Here also, gripping strength can be controlled by the amount of electron-beam energy that is used to cross-link the gel. The gel may peel away from the denture and oral tissues cleanly, without residue.

The detailed description set forth hereinbelow is intended as a description of a variety of exemplary hydrogel compositions provided in accordance with one or more aspects of the present invention and is not intended to represent the only forms which may be prepared or utilized. The description sets forth features and/or operations for preparing and using hydrogel compositions according hereto. It is to be understood, however, that the same or equivalent functions and ingredients incorporated in the hydrogel compositions hereof may be accomplished by different embodiments that are nevertheless also intended to be and are encompassed within the spirit and scope of the present invention.

### DETAILED DESCRIPTION

The present invention is directed generally to new hydrophilic cohesive hydrogel products which are adapted for a variety of oral uses; e.g., from form-fitting sticky denture fixatives or alveolitis packing to controlled release devices for deliver of bioactive materials.

In some implementations of the present invention, a hydrophilic oral and dental cohesive gel sheet or device capable of securing and cushioning dental prostheses for humans or other animals may be formed and used. In particular, a variety of denture fixatives, including alternative liners and/or powders may be used.

Such developments include a bio-adhesive, high-water content denture liner/fixative that may dissolve very slowly, flow to distribute occlusal forces, cushion to minimize denture sores and feels smooth, non-gummy, and non-greasy. Up to about 2% gum or alginate may be added to the ingredients to supplement adhesion. The gums and alginates may also be powder coated on the outside of an intermediate integral outer thin paper or woven sheet.

The fixative devices hereof may be provided in a variety of denture-approximating shapes, as well as thin strips and circles and may be supplied sealed in airtight, heat-sealed pouches and in contact with a release sheet, for example a sheet of plastic or coated plastic (e.g. siliconised plastic) or paper or coated paper (e.g. siliconised paper). The denture fixative liners may have very good scissors-cutting performance and may easily be trimmed by the end user to create a perfect fit within the internal mucosal surface of the denture.

The resulting denture fixative liners may cause superior retention of dentures by cohering to porous denture and mucosal surfaces by having low surface tension yet high structural integrity. Additionally when leakage under the denture occurs, the liner is hydrophyllic and may swell to fit thereby causing a friction luting effect.

For increased retention, the side of the liner intended to contact the denture could have an outer paper or woven coating. For even further enhanced retention, the outer paper coating could be painted with an orally compatible liquid paint-on mucosal bonding agent such as 2-octyl cyanoacrylate. If used in this manner the hydrogel liner fixative would typically be applied no more than about 1-2 times daily.

The hydrogel base ingredients may include water (between about 90% and about 95%); a hydrogel polymer (about 3%) selected from one or more of polyacrylamide, polyethylene glycol, polyvinylpyrrolidone, carboxymethyl cellulose and an adhesivity agent (about 3%) selected from xanthan gum and sodium alginate. Other additives and/or water may make up the remainder.

The manufacture and cross-linking of the hydrogels hereof may be the result of using an electron beam technology. (An example is available from Hydrogel Design Systems, Inc., Langhorne, Pa., USA.) There may either be no internal scrim or there may be a blue plastic netting so that the hydrogel can easily be seen. Such a scrim may be a light open texture, usually made of cotton or flax, as may typically be used in bookbinding, upholstery and other industries or as used as a backing to strengthen paper, as in maps and packing paper.

Various additives are possible, including: nutritive, flavoring, sweetening and/or coloring agents; antimicrobial, antiseptic and/or antifungal agents; numbing agents (for persons with denture sores); gums or alginates to increase adhesion; calcium to be released slowly during the daily use to help prevent osteoporosis. Other possible additives may include breath freshening agents, saliva stimulating agents.

Breath Freshening Agents used in oral care products are typically sulfur precipitating agents. Sulfur precipitating agents bind with and inactivate the volatile sulfur compounds that cause a large percentage of oral malodor. Those which may be useful with the denture fixatives hereof include metal salts such as copper salts and zinc salts. Preferred salts include copper gluconate, zinc citrate and zinc gluconate. The amount of sulfur precipitating agent is from about 0.5% to about 1.0 wt %. Saliva stimulating agents may be used and may include food acids such as citric, lactic, malic, succinic, ascorbic, adipic, fumaric and tartaric acids. Preferred food acids are citric, malic and ascorbic acids. The amount of saliva stimulating agents in the film may be from about 2% to about 4%.

As introduced above, antimicrobial and/or antiseptic agents may also or alternatively be used. Suitable antimicrobial and antiseptic agents can be included individually or in combination in order to synergistically be effective in killing the plaque-producing bacteria that cause dental plaque, gingivitis and/or bad breath. Orally compatible antiseptic and antimicrobial agents are well known in the art and include: essential oils and/or CPC Antiseptics. As to the essential oils, two or more may be used in the flexible high-water content hydrogel strips composition can vary as long as they are in amounts sufficient to provide antimicrobial efficacy. Three typically used essential oils include thymol, methyl salicylate and eucalyptol. These can each be added to hydrogel strips from about 0.75 to about 2.0 wt %. Another essential oil, menthol can be added from about 2% to about 5% and also gives the sensation of a cooling. As to the CPC Antiseptics, Cetylpyridinium chloride (CPC) is a cationic quaternary ammonium compound that has been shown to possess antimicrobial activity, and/or provide control of dental plaque. When formulated appropriately, CPC is also effective at reducing gingivitis. A combination of Cetylpridinium chloride about 0.025 to about 0.1 wt % and Domiphen Bromide (about 0.0002 wt %) is used in many mouthwash preparations and can also be used in the herein described hydrogel denture fixative strips.

Sweetening Agents may be used as well. Suitable non-cariogenic artificial sweeteners that can be included are those well known in the art. These include: water-soluble artificial sweeteners such as the soluble saccharin salts; dipeptide based sweeteners, such as L-aspartic acid derived sweeteners, such as L-aspartyl-L-phenylalanine methyl ester (aspartame) and materials described in U.S. Pat. No. 3,492,131; water-soluble sweeteners derived from naturally occurring water-soluble sweeteners, such as a chlorinated derivative of ordinary sucrose known under the product description of sucralose; and protein based sweeteners such as thaumatoccous danielli (Thaumatin I & II). In general, an effective amount of auxiliary sweetener may be utilized to provide the level of sweetness desired for a particular composition, and this amount will vary with the sweetener selected. About 1 to about 6 wt % is most preferred. Note, it is preferable to avoid adding both copper gluconate and saccharin at the same time to the aqueous solution, as a precipitate will form. Thus, it is preferred to combine sweeteners other than saccharin with copper gluconate.

Flavoring Agents may also/alternatively be used. Commonly used flavors (up to about 2%) include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in “Chemicals Used in Food Processing,” publication 1274 by the National Academy of Sciences, pages 63-258, may be used.

Nutritive Agents may also be used. In one example, up to 500 mg of calcium carbonate may be optionally added as a nutritive agent. As the denture fixative liner dissolves through the day, a slow- released stream of healthful calcium may be swallowed or otherwise be transmitted, e.g., via absorption through the gums, and thus delivered to the body for the prevention of osteoporosis.

The thickness of the hydrogel sheets and shapes hereof may be of a substantially uniform thickness of about two hundredths of an inch (0.02″) to about twenty-three hundredths of an inch (0.23″). The shape and/or dimensions may include: A) a shape approximating the inner surface of an upper or lower denture; and/or B) a collection of shapes including: a) about 8 mm wide by about 4 inch long strip with three (3) V-notches located on one edge at about 1 inch intervals to allow for bending (for lower denture or upper denture post-dam area); b) about 8 mm diameter circles (for lower denture); c) short strips that are about 8 mm wide and about 24 mm long (for either upper or lower denture); d) about 20 mm by about 10 mm oval islands for upper dentures.

As an external coating, either A) no coating may be used (in which case, the end user may optionally apply a methylcellulose liquid gel adhesive to one or both sides for added retention); B) xanthan gum or sodium alginate thin powder layer added at time of manufacture or added as an adhesion supplement by the end user; or, C) a porous paper or woven remay liner integrated on the outer surface of one side, may be used.

For an external liner; plastic; siliconised plastic; paper or coated paper may be used. The adhesive liners are island placed between two liner sheets can be wound about itself (e.g., around a spool), These non-stick liners are either perforated and z-folded—or cut between each strip and stacked in order to prevent adjacent portions of hydrogel strips from sticking together.

For packaging; mylar, plastic, polyethylene or siliconized plastic or the like may be used. Airtight crimped or heat sealed pouches to prevent loss of moisture. This may optionally be contained with a plastic hinged top denture bath container with removable inner casement. Once the inner pouch is opened, a few drops of water can be added inside the denture bath container to maintain humidity and moistness of the gels, so long as the top of the bath is closed.

A tube of a liquid gel adhesive including about 95% water, about 4% methylcellulose and about 1% xanthan gum, with about 0.003% FD&C red coloring may be included for the end user to apply on the surface of the hydrogel to increase adhesion. Alternatively a sprinkle bottle containing xanthan gum or sodium alginate may be included for the end user to powder coat the hydrogel at the time of use—again for the purpose of increasing adhesion.

An exemplar implementation is set forth in the table below:

DENTURE COMPOSITION #1

| Water | 90-91% QS |
|---|---|
| Hydrogel Polymer | 3% |
| Xanthan Gum or Sodium Alginate | 3% |
| Mint Flavor | 2% |
| Sucralose | 0.75% |
| Calcium carbonate | 1% |
| Total | 100% |

A representation of two alternative shaped hydrogels structures is presented here.

[image]

In a further set of implementations, the present invention may also and/or alternatively provide a treatment for alveolitis (post-extraction dry socket). In some instances, this may involve a novel one-step, swellable, sterile, self-eliminating treatment for acute alveolitis which rapidly alleviates pain and provides a cooling, soothing effect throughout the healing process. Its swell-to-fit, tacky gel consistency allows for easy filling of the socket and good adherence during the entire healing process. The active ingredients included within the high water content gel strips may include eugenol for analgesic action, butamben for anesthetic action, and a mixture of four essential oils for cooling antimicrobial action.

A high-water content, non-chemical cross-linked, high-energy electron-beam cross-linked hydrogel may be specially beneficial for use in treatment for alveolitis. In particular, such a hydrogel may have the following unique characteristics: 3-dimensional swellable; bioabsorbable; sterile as a result of the high energy electron-beam cross-linking; is cooling; microbe impermeable; oxygen permeable; has no chemical cross linkers; and is mostly water. Such a hydorgel may be supplied in sealed sterile pouches containing several, in some cases, four, hydrogel strips each. The dimensions of the hydrogel strips may be about 1 inch long and about ¼ inch wide. To maintain moisture, the strips may be heat sealed into airtight pouches with a small amount (about 5 ml) of surrounding liquid storage medium.

Such a device may be advantageous over current therapies in that high-water content, electron beam cross-linked hydrogels are materials which absorb water, blood and saliva; undergo rapid swelling without discernible dissolution; and may maintain three-dimensional networks capable of reversible deformation over time. This particular form of hydrogel may be virtually chemical-free being cross-linked with an electron-beam instead which can give the end user a soothing, cool feeling. Additionally it is orally compatible, edible, non-toxic, and it may be capable of slow releasing an active ingredient. Most importantly, because of its nature of being cross-linked with high-energy electron beam, it is substantially sterile, if not 100% sterile when packaged and sealed into an airtight pouch. As it is bioabsorbable, the dentist need not retrieve it as is the case with most other alveolitis treatments. It also has the “swell to fit” advantage, immediately taking on the three dimensional morphology of the open tooth socket without needing to apply painful pressure during packing into the open socket.

The hydrogel base ingredients hereof may include water (about 70%); a hydrogel polymer (about 3%) selected from polyacrylamide, polyethylene glycol, polyvinylpyrrolidone, carboxymethyl cellulose with an anesthetic and/or an analgesic (about 22% to about 23.5%) such as eugenol about 13.5% and butamben about 10%; and one or more antimicrobial agents (combined, about 2.5%) such as thymol about 0.5%, methyl salicylate about 0.5%, eucalyptol about 0.5%, menthol about 1%; and finally a flavoring and/or coloring to about 2.5% (see below).

The storage liquid ingredients (about 5 ml per pouch) may include water at about 73%; polymer about 0%; all other ingredients about 27% as above. Active ingredients and other additives for the storage liquid may include: anesthetic and/or analgesic (about 23.5%); eugenol about 13.5%; butamben about 10%; antimicrobial agents (combined about 5%); Thymol about 1%; methyl salicylate about 1%; eucalyptol about 1%; menthol about 2%; Flavoring Agents (about 2%) Commonly used flavors (up to about 2%) include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in Chemicals Used in Food Processing, publication 1274 by the National Academy of Sciences, pages 63-258, may be used. Coloring Agents (less than about 0.5%), the coloring agents useful in the present invention, include pigments such as titanium dioxide, which may be incorporated in amounts less than about 1 wt %. Colorants can also include natural food colors and dyes suitable for food, drug and cosmetic applications. These colorants are known as FD&C dyes and lakes. The materials acceptable for the foregoing spectrum of use are preferably water-soluble, and include FD&C Blue No. 2 and Green No. 3 (A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.)

Manufacture and cross-linking of the gels may be the result of using an electron beam technology. The internal scrim may be none or the thickness may be uniform thickness of about two hundredths of an inch (0.02″) to about twenty-three hundredths of an inch (0.03″). Shape and/or Dimensions rectangular strips 1″ long by ¼″ wide. There may be no external coating. There may be no external liner. For packaging, several strips (e.g., four (4) strips) per application may be stored in liquid-filled mylar, plastic, polyethylene or siliconized plastic pouches that are airtight and crimped or heat sealed pouches to prevent loss of moisture.

An exemplar composition for treating alveolitis is shown here.

HYDROGEL COMPOSITION

| | |
|---|---|
| Water | 70% |
| Hydrogel Polymer | 3% |
| Eugenol Anesthetic | 12% |
| Bupamben Analgesic | 10% |
| Menthol USP | 1.00% |
| Methyl Salicylate | 0.50% |
| Eucalyptol | 0.50% |
| Thymol | 0.50% |
| Mint Flavor | 2% |
| Titanium Dioxide | 0.5% |
| Total | 100% |

An exemplar composition for containing such a hydrogel is shown here.

STORAGE LIQUID COMPOSITION

| | |
|---|---|
| Water | 73% |
| Eugenol Anesthetic | 12% |
| Bupamben Analgesic | 10% |
| Menthol USP | 1.00% |
| Methyl Salicylate | 0.50% |
| Eucalyptol | 0.50% |
| Thymol | 0.50% |
| Mint Flavor | 2% |
| Titanium Dioxide | 0.5% |
| Total | 100% |

A representation of two alternative shaped hydrogels structures is presented here.

[image]

Slow-released, slowly-swallowed, smoking cessation and appetite control integrated system. A tooth whitening bioadhesive hydrogel may be provided, e.g., in an over the counter (OTC) high concentration form. Tooth whitening hydrogels of this sort may be bioadhesive dots allowing for good containment and activated substantially continuous release of peroxygen bleaching agent and/or for oral malodors.

The present invention relates to a controlled release system useful for delivery of biologically active ingredients smoking cessation and/or subsequent appetite control over an extended period of time. Many persons desire a gradual method for withdrawal from their nicotine addiction. Because smoking cessation is often followed by weight gain, an optional integrated appetite control gel strip may be included in the system for use after the complete withdrawal from nicotine. In both cases (smoking cessation and appetite control), the delivery mechanism may be through a high water content, non-chemical but rather electron-bean cross-linked, flexible, edible, tacky hydrogel strip that may be particularly well adapted to adhere to the soft tissues of the oral cavity and slowly dissolve in the mouth of a consumer.

The slow dissolving hydrogel strips deliver an orally compatible and digestible appetite control or nicotine control agent. Additionally, the slow dissolving hydrogel strips contain a sweetener, flavored breath freshening agent and a coloring agent and may also optionally contain antiseptic or antimicrobial ingredients.

The hydrogels strips are flexible and tacky, between about 0.010″ to about 0.100″ thick, about ¼″ to ½″ rectangular and slowly release the oral care agents in the oral cavity while slow dissolving to provide extended efficacy.

About 5% to about 30% Pullulan, Cellulose Alpha Starch or Glucan (or other polysaccharide) polymer may be added to the previously claim base ingredients to increase tackiness and to control the rate of dissolution. An additional about 1% to about 3% Xanthan gum may also be added to increase adhesion.

In order to prevent local transdermal action (delivery mechanism is via intraoral route—not transdermal) and to increase mucosal adhesion, two additional layers are integrated onto the hydrogel sheets prior to cutting into oval shapes. The first is a polymer coating known to those skilled in the art. These include polymers such, as Eudragit E. cellulosics, such as ethylcellulose, and the like.

The outermost layer is comprised of an integrated porous paper liner that will come into contact with the mucosal surface. This paper liner increases adhesion, especially when used in combination with a liquid paint-on mucosal bonding agent such as 2-Octyl Cyanoacrylate. If used in this manner the hydrogel would be applied; generally, no more than 3-4 times daily.

The smoking cessation hydrogel strip slow delivers over about 2 to about 4 hours either about 6.0, about 3.0 or about 1.5 mg of nicotine (about 2 mg/hr to about 0.5 mg/hr) as a substitute for smoking a cigarette. During the first week the consumer uses the highest dosage level strips. Each week, the next lower dosage strip is used. After about 3 weeks, no strips are used. Thus the invention provides a three step, 3-week gradually declining dosage of nicotine to allow for lessened nicotine cravings after a person decides to stop smoking. This gradual approach is preferable by many people over the “cold-turkey” approach and research has shown that persons using a gradually declining mechanism for delivery of nicotine during the immediate three weeks after a decision to cease smoking cigarettes leads to a higher permanent success rate. The invention is preferable over conventional transdermal patches as it is intraoral and not visible. It is preferable over nicotine gums, as gum chewing is often considered socially unacceptable.

The appetite control strip is sold in combination with the nicotine control strip, because many people tend to gain weight during the 3-4 weeks following smoking cessation. These strips help to prevent weight gain following complete nicotine withdrawal. After the nicotine control strips are used, the consumer has the option of using the appetite control strips for 3 weeks. These strips contain benzocaine that is slow released to negatively alter and diminish taste sensation and thus decrease appetite. Furthermore a fat absorption blocker is added to bind fats of any consumed foods and reduce fat absorption into the bloodstream. This kind of additive has shown to be successful in weight maintenance programs.

The hydrogel base ingredients include water (about 70% to about 90%) and a Polymer/Polysaccharide (about 10%-about 30%) selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. A preferred embodiment may contain about 3% polyvinyl pyrrolidone, about 1-2% Xanthan Gum or Sodium Alginate, and about 5% - about 20% Pullulan.

Alternative active ingredient additives may include a smoking cessation aid of Nicotine; e.g., about 3.00 mg per strip (3x daily), and/or an appetite control aid which may provide an alteration of taste, e.g., Benzocaine at about 5% (3x daily). Moreover, a combination may be made with a fat absorption blocker, e.g., Orlistat (silfosafen) about 50 mg.

Sweetening Agents may be used as well. Suitable non-cariogenic artificial sweeteners that can be included are those well known in the art. These include: water-soluble artificial sweeteners such as the soluble saccharin salts; dipeptide based sweeteners, such as L-aspartic acid derived sweeteners, such as L-aspartyl-L-phenylalanine methyl ester (aspartame) and materials described in U.S. Pat. No. 3,492,131; water-soluble sweeteners derived from naturally occurring water-soluble sweeteners, such as a chlorinated derivative of ordinary sucrose known under the product description of sucralose; and protein based sweeteners such as thaumatoccous danielli (Thaumatin I & II). In general, an effective amount of auxiliary sweetener is utilized to provide the level of sweetness desired for a particular composition, and this amount will vary with the sweetener selected. About 1 to about 6 wt % is most preferred. Note, it is preferable to avoid adding both copper gluconate and saccharin at the same time to the aqueous solution, as a precipitate will form. Thus, it is preferred to combine sweeteners other than saccharin with copper gluconate.

Flavoring Agents may also/alternatively be used. Commonly used flavors (up to about 2%) include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in “Chemicals Used in Food Processing,” publication 1274 by the National Academy of Sciences, pages 63-258, may be used.

Nutritive Agents may also be used. In one example, up to about 500 mg of calcium carbonate may be optionally added as a nutritive agent. As the denture fixative liner dissolves through the day, a slow-released stream of healthful calcium may be swallowed or otherwise be transmitted, e.g., via absorption through the gums, and thus delivered to the body for the prevention of osteoporosis.

The manufacture and cross-linking of the gels may be the result of using unique proprietary technology utilizing an electron beam. Few machines capable of creating this gel exist in the world. One such machine is available from Hydrogel Design Systems, located in Langhorne, Pa., USA.

There may be but typically will be no internal scrim. Thickness may be uniform thickness of about twenty to twenty-five thousandths of an inch (about 0.02″ about 0.025″). Shape and/or Dimensions; Oval intraoral patches about 8 mm by about 4 mm.

As manufactured, an external coating may be coated on mucosal surface contact side with a porous paper liner. Between the gel and the porous paper liner, the gel has a coating to prevent local delivery of numbing anesthetic or nicotine. The delivery is via a swallowing mechanism, not transdermal. The coating used to prevent transdermal local delivery of the active ingredients are coatings known to those skilled in the art. These include polymers such, as Eudragit E, cellulosics, such as ethylcellulose, and the like.

For an external liner; on one or both sides of: plastic, siliconised plastic, paper or coated paper. The ovals are island placed on liner sheets which can be wound about themselves (e.g., around a spool). These non-stick liners are either perforated and z-folded—or cut between each strip and stacked in order to prevent adjacent portions of hydrogel ovals from sticking together. For packaging; mylar, plastic, polyethylene or siliconized plastic. Airtight crimped or heat scaled pouches to prevent loss of moisture. This may optionally be contained with a plastic hinged top denture bath container with removable inner casement. Once the inner pouch is opened, a few drops of water can be added inside the denture bath container to maintain humidity and moistness of the gels, so long as the top of the bath is closed.

A tube of liquid paint-on mucosal bonding agent such as 2-Octyl Cyanoacrylate is supplied with an applicator brush. This is used to firmly adhere the hydrogel to the mucosa so that up to about 6 hours of slow release activity can occur without dislodging the hydrogel ovals.

SMOKING CESSATION HYDROGEL COMPOSITION

| | |
|---|---|
| Water | 68-75% |
| Pululan | 10% |
| Hydrogel Polymer | 5% |
| Nicotine | 3%-10% |
| Flavor | 2% |
| Menthol USP | 2.00% |
| Xanthan Gum | 0.40% |
| Citric Acid | 1% |
| Sucralose | 0.75% |
| Total | 100% |

APPETITE SUPPRESSION HYDROGEL COMPOSITION

| | |
|---|---|
| Water | 49% |
| Pululan | 10% |
| Silfosafen | 25% |
| Hydrogel Polymer | 5% |
| Benzocaine | 5% |
| Flavor | 2% |
| Menthol USP | 2.00% |
| Xanthan Gum | 0.40% |
| Citric Acid | 1% |
| Sucralose | 0.75% |
| Total | 100% |

One surface is overlayed with 5% ethylcellulose coated porous paper liner

This invention also relates to improvements in tooth whitening technology. OTC High Concentration tooth whitening bio-adhesives dots allowing for superior containment and activated continuous release of peroxygen bleaching agent.

The present invention relates to a novel end-user activated, controlled release system useful for bleaching teeth o with a superior ability to contain and restrict high concentrations of peroxigens or nitrogen oxide analogs to tooth surfaces and allowing only very low concentrations to be detected elsewhere in the oral cavity. Additionally, the consumer applied activator adhesive causes whitening of teeth to occur rapidly. The delivery mechanism is a series of about 6 to about 10 (per dental arch) high water content, non-chemical but rather electron-beam cross-linked, flexible, orally compatible, tacky hydrogel oval dots that are particularly well adapted to adhere to the teeth and slowly dissolve over the course of about 15 to about 30 minutes. The dots can then be removed and discarded by the consumer at will.

The slow dissolving hydrogel dots deliver high concentration dosage directly onto the surface of teeth. Additionally, the slow dissolving hydrogel dots contain a sweetener and a flavored breath freshening agent.

The high water content, thin hydrogel dots are sandwiched between a coating of two thin woven cotton remay or paper liners on both sides. The middle layer is the electron-beam cross-linked high concentration peroxide-containing hydrogel. The peroxide is stabilized in the gel by adjusting the pH to about 2.3 and therefore will not become active for tooth bleaching until adhered onto each tooth with a pH=about 10.8 liquid applied from a hollow-filled swab. Once placed on the teeth, the non-tooth contacting side of the hydrogel dot is coated with a flavored vitamin E oil lubricant applied with a swab. This essentially blocks the peroxide from being released intraorally and prevents the gel dots from adhering to the labial and vestibular mucosal surfaces.

The ovals are of various sizes to accommodate different sized teeth. Each outer coating sheet is about 0.10″ thick and the hydrogel sheet is about 0.023″ thick. The total thickness is approximately 0.040″ which is the same thickness of well-tolerated professionally fabricated custom trays and should not interfere with speech or other oral functions.

Because this method allows for a higher concentration of hydrogen peroxide to be delivered to the tooth, and because the tooth adhering agent is a powerful peroxide activator, and because the mechanism contains the caustic, mucosal-irritating peroxigen to the surface of teeth only, teeth become about 6 to about 11 shades whiter after only six daily applications. The invention is extremely thin and comfortable, pleasantly flavored, relatively easy to apply and is safe when used as directed.

Furthermore a light activator is optionally included in the formulation which further enhances the activity of the gel dots, if the consumer uses an optionally supplied cheek and lip retractor and exposes the teeth to an optionally included low wattage led light or even a regular incandescent flashlight desk light. When exposing teeth to light, eye protection should be worn.

Optionally, The high water content, electron-beam cross-linked dots can be substituted with a single about 2″-about 3″ long×about ¼″ wide strip (per arch)

The hydrogel base ingredients here may include water (about 75%); hydrogen peroxide (about 16%); and one or more hydrogel polymers (about 15%) selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. A preferred embodiment may contain about 5% polyvinyl pyrrolidone, about 1% xanthan gum or sodium alginate, and about 9% pullulan. Optionally also includable are flavoring (2%) and/or sweetener (about 2%). In any case, the pH may be adjusted below about 5.0 with citric or tartaric acid (preferably below about 3.0).

Possible additives hereof may include a tooth adhesive high pH activator gel to be applied to surface of outer paper or woven liner that will be in contact with teeth. The gel is formulated with >90% water, 2% xanthan gum, 2% ethyl or methyl cellulose, <2% baking, soda, <2% sodium carbonate and sufficient potassium hydroxide to above pH=10.0 (preferably pH=10.8). Optionally <1.0 wt % of octylcyanoacrylate can be added to further enhance tooth retention. This solution can be painted on each gel dot using a supplied brush or dispensed from a hollow-filled cotton swab delivery mechanism.

Other possible additives hereof may include aloe vera and/or a vitamin F oil lubricant may be applied to outer surface paper liner to come in contact with oral soft tissues. Prevent the dots from sticking to the inner lips and cheeks and provides soothing aloe vera oil and antioxidant vitamin E oil to add extra comfort & safety during treatment. The ingredients are greater than about 80% silicone lubricant, aloe vera oil about 10 wt %, vitamin E oil about 1 %-about 2%, flavoring about 2%, sweetener about 2% and color about 0.003%.

Optional sweetening agents may be included for a gel or outer liner. Suitable non-cariogenic artificial sweeteners that can be included are those well known in the art. These may include: A. water-soluble artificial sweeteners such as the soluble saccharin salts; B. dipeptide based sweeteners, such as L-aspartic acid derived sweeteners, such as L-aspartyl-L-phenylalanine methyl ester (aspartame) and materials described in U.S. Pat. No 3,492,131; C. water-soluble sweeteners derived from naturally occurring water-soluble sweeteners, such as a chlorinated derivative of ordinary sucrose known under the product description of sucralose; and D. protein based sweeteners such as thaumatoccous danielli (Thaumatin I & II).

In general, an effective amount of auxiliary sweetener is utilized to provide the level of sweetness desired for a particular composition, and this amount will vary with the sweetener selected. About 1 to about 6 wt % being most preferred.

OPTIONAL FLAVORING AGENT For gel or outer liner. Commonly used flavors (up to 2%) include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also he used. (Generally, any flavoring or food additive, such as those described in Chemicals Used in Food Processing, publication 1274 by the National Academy of Sciences, pages 63-258, may be used.)

OPTIONAL COLORING AGENT For outer liner lubricant only. The coloring agents useful in the present invention, include pigments such as titanium dioxide, which may be incorporated in amounts <1 wt %. Colorants can also include natural food colors and dyes suitable for food, drug and cosmetic applications. These colorants are known as FD&C dyes and lakes. The materials acceptable for the foregoing spectrum of use are preferably water-soluble, and include FD&C Blue No. 2 and Green No. 3 (A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.)

The manufacture and cross-linking of the gels may be the result of using unique proprietary technology utilizing an electron beam. Only two machines capable of creating this gel exist in the world. One such machine is available from Hydrogel Design Systems, Inc., a subsidiary of Nesco Industries Inc., Hydrogel Design Systems, Inc., being located in Langhorne, Pa., USA.

INTERNAL SCRIM blue plastic netting embedded within. The thickness may be uniform thickness of about twenty to twenty-five thousandths of an inch (0.02″- 0.025″). SHAPE/DIMENSIONS Oval dots—3 sizes; 12 mm×6 mm, 10 mm×5 mm, 8 mm×4 mm; or a SINGLE STRIP 2″ to 3″ long×10 mm wide. As manufactured, an external coating may be coated on each side with a porous paper liner.

AS ADDED BY CONSUMER Liner towards tooth surface is further coated by end user with adhesive gel containing high pH activator, methylcellusose, xanthan gum and optionally less than about 1% 2-octyl-cyanoacrylate.

Exterior liner is integrated plastic or coated paper made further impermeable, soothing and safer by coating with silicone-based oil with aloe vera oil, vitamin E oil, flavoring and sweetener. No external liner is needed. The hydrogel is sandwiched between integrated porous paper liners.

On one or both sides of: plastic, siliconised plastic, paper or coated paper. The ovals are island placed on liner sheets which can be wound about themselves (e.g., around a spool). These non-stick liners are either perforated and z-folded—or cut between each strip and stacked in order to prevent adjacent portions of hydrogel ovals from sticking together.

For packaging, A number of dots (e.g., 30 various shaped) may be filled into daily use pouches or aligned on a cardboard strip. Each daily dose is sealed within mylar, plastic, polyethylene or siliconized plastic and airtight crimped or heat sealed pouches to prevent loss of moisture.

HYDROGEL COMPOSITION

| | |
|---|---|
| Water | 75% |
| Hydrogen Peroxide | 16% |
| Pululan | 9% |
| PVP Polymer | 5% |
| Xanthan Gum | 1% |
| Menthol USP | 2.00% |
| Mint Flavor | 2% |
| Citric Acid | 1% |
| Sucralose | 0.75% |
| Total | 100% |

See above for outer adhesive and outer lubricant composition

Optionally supplied with lip and check retractor, led light, eye protection.

The invention provides a high water content, non-chemical but rather electron-beam cross-linked, flexible, edible, tacky hydrogel strip that is particularly well adapted to adhere to the soft tissues of the oral cavity and slowly dissolve in the mouth of a consumer. The time to completely dissolve the hydrogel strip is > about 15 minutes and < about 13 hours.

The slow dissolving hydrogel strip delivers at least one oral care agent, such as breath freshening or antimicrobial. The antimicrobial agents are effective against bacteria that cause halitosis, dental plaque, and gingivitis. The salivary stimulants are effective against the condition known as xerostomia or dry mouth. Additionally, the slow dissolving hydrogel strips may contain a mint or cinnamon flavored breath freshening agent alone or in combination with the antimcrobial ingredients. Finally, sulfur precipitating agents that reduce oral malodor can also be added to the slow dissolving gel strips according to the present invention. These agents bind with, and inactivate, the volatile sulfur compounds that cause a large percentage of oral malodor.

The hydrogel strips are flexible and tacky, between about 0.010″ to about 0.100″ thick, ¼″ to ½″ rectangular and slowly release the oral care agents in the oral cavity while slow dissolving to provide extended efficacy.

5%-30% pullulan, cellulose alpha starch or glucan (or other polysaccharide) polymer is added to the previously claim base ingredients to increase tackiness and to control the rate of dissolution. 1% to 3% Xanthan gum may also be added to increase adhesion. Additional ingredients may include coloring, sweetener, mint flavor and antimicrobial agent(s) as described further below.

The hydrogel base ingredients may include water (70% to about 90%); polymer/polysaccharide (10% - 30%) selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof The preferred embodiment may contain 3% polyvinyl pyrrolidone, 1-2% xanthan gum or sodium alginate, and 5%-20% pullulan. An internal scrim or plastic netting may be used, though is not necessary, the scrim making the hydrogel easily seen in a preferred embodiment.

The manufacture and cross-linking of the gels may be the result of using unique proprietary technology utilizing an electron beam. It currently appears that only two machines capable of creating this gel exist in the world. One such machine is available from Hydrogel Design Systems, Inc., a subsidiary of Nesco Industries Inc., Hydrogel Design Systems, Inc., being located in Langhorne, Pa., USA.

POSSIBLE ADDITIVES include Breath Freshening Agents, saliva stimulating agents, antimicrobial and antiseptic agents, sweetening, flavoring and/or coloring agents, and/or nutritive agents.

Breath Freshening Agents used in oral care products are typically sulfur precipitating agents. Sulfur precipitating agents bind with and inactivate the volatile sulfur compounds that cause a large percentage of oral malodor. Those useful with the denture fixatives hereof include metal salts such as copper salts and zinc salts. Preferred salts include copper gluconate, zinc citrate and zinc gluconate. The amount of sulfur precipitating agent is from about 0.5% to about 1.0 wt %.

Saliva stimulating agents may be used and may include food acids such as citric, lactic, malic, succinic, ascorbic, adipic, fumaric and tartaric acids. Preferred food acids are citric, malic and ascorbic acids. The amount of saliva stimulating agents in the film may be from about 2% to 4%.

Antimicrobial & Antiseptic Agents may also be used. Suitable antimicrobial and antiseptic agents can be included individually or in combination in order to synergistically be effective in killing the plaque-producing bacteria that cause dental plaque, gingivitis and bad breath. Orally compatible antiseptic and antimicrobial agents are well known in the art and include: essential oils and/or CPC Antiseptics. As to the essential oils, two or more may be used in the flexible high-water content hydrogel strips composition can vary as long as they are in amounts sufficient to provide antimicrobial efficacy. Three typically used essential oils include thymol, methyl salicylate and eucalyptol. These can each be added to the hydrogel strips from 0.75 to about 2.0 wt %. Another essential oil, menthol can be added from about 2% to 5% and also gives the sensation of a cooling. As to the CPC Antiseptics, Cetylpyridinium chloride (CPC) is a cationic quaternary ammonium compound that has been shown to possess antimicrobial activity, and/or provide control of dental plaque. When formulated appropriately, CPC is also effective at reducing gingivitis. A combination of Cetylpridinium chloride 0.025 to 0.1 wt % and Domiphen Bromide (0.0002 wt %) is used in many mouthwash preparations and can also be used in the herein described hydrogel denture fixative strips.

Sweetening Agents may be used as well. Suitable non-cariogenic artificial sweeteners that can be included are those well known in the art. These include: water-soluble artificial sweeteners such as the soluble saccharin salts; dipeptide based sweeteners, such as L-aspartic acid derived sweeteners, such as L-aspartyl-L-phenylalanine methyl ester (aspartame) and materials described in U.S. Pat. No. 3,492,131); water-soluble sweeteners derived from naturally occurring water-soluble sweeteners, such as a chlorinated derivative of ordinary sucrose known under the product description of sucralose; and protein based sweeteners such as thaumatoccous danielli (Thaumatin I & II). In general, an effective amount of auxiliary sweetener is utilized to provide the level of sweetness desired for a particular composition, and this amount will vary with the sweetener selected. About 1 to about 6 wt % is most preferred. Note, it is preferable to avoid adding both copper gluconate and saccharin at the same time to the aqueous solution, as a precipitate will form. Thus, it is preferred to combine sweeteners other than saccharin with copper gluconate.

Flavoring Agents may also/alternatively be used. Commonly used flavors (up to 2%) include mints such as peppermint, artificial vanilla, cinnamon derivatives, and various fruit flavors, whether employed individually or in admixture. Flavorings such as aldehydes and esters including cinnamyl acetate, cinnamaldehyde, citral, diethylacetal, dihydrocarvyl acetate, eugenyl formate, p-methylanisole, and so forth may also be used. Generally, any flavoring or food additive, such as those described in “Chemicals Used in Food Processing,” publication 1274 by the National Academy of Sciences, pages 63-258, may be used.

Coloring agents may be useful or preferred for a variety of reasons in the present invention, include use of pigments such as titanium dioxide, which may be incorporated in amounts less than or equal to about 1 wt %. Colorants can also include natural food colors and dyes suitable for food, drug and cosmetic applications. These colorants are known as FD&C dyes and lakes. The materials acceptable for the foregoing spectrum of use are preferably water-soluble, and include FD&C Blue No. 2 and Green No. 3. (A full recitation of all FD&C and D&C dyes and their corresponding chemical structures may be found in the Kirk-Othmer Encyclopedia of Chemical Technology, Volume 5, Pages 857-884, which text is accordingly incorporated herein by reference.)

Nutritive Agents may also be used. In one example, up to 500 mg of calcium carbonate may be optionally added as a nutritive agent. As the denture fixative liner dissolves through the day, a slow-released stream of healthful calcium may be swallowed or otherwise be transmitted, e.g., via absorption through the gums, and thus delivered to the body for the prevention of osteoporosis.

The manufacture and cross-linking of the gels may be the result of using unique proprietary technology utilizing an electron beam. Only two machines capable of creating this gel exist in the world. One such machine is available from Hydrogel Design Systems, Inc., a subsidiary of Nesco Industries Inc., Hydrogel Design Systems, Inc., being located in Langhorne, Pa., USA.

INTERNAL SCRIM None—or—Blue plastic netting so that the hydrogel is easily seen (preferred)

Thickness may be uniform thickness of about two hundredths of an inch (0.05″) to about twenty-three hundredths of an inch (0.1″). The thicker the get, the longer-lasting and more slow dissolving the strip will be.

The shape and/or dimensions of some particular embodiments may include: rectangles of up to ⅜″ wide and/or up to ½″ long; or, circles of up to ½″ diameter.

No external liner will be necessary; however, for an external liner, plastic, siliconised plastic, paper or a coated paper may be used. The adhesive liners are island placed between two liner sheets can be wound about itself (e.g., around a spool), These non-stick liners are either perforated and z-folded—or cut between each strip and stacked in order to prevent adjacent portions of hydrogel strips from sticking together.

For packaging, either mylar, plastic or siliconized plastic may be used. Airtight crimped or heat sealed pouches. Small plastic containers with semi-airtight hinged lids may be used to dispense the hydrogel strips, but even in this case the container should be sealed within airtight sealed pouches to prevent loss of moisture content.

PREFERRED COMPOSITION #1

| | |
|---|---|
| Water | 75% |
| Pululan | 10% |
| Hydrogel Polymer | 5% |
| Xanthan Gum | 0.40% |
| Thymol | 1% |
| Menthol USP | 2.00% |
| Methyl Salicylate | 0.50% |
| Eucalyptol | 0.50% |
| Mint Flavor | 3% |
| Copper Gluconate | 0.40% |
| Sucralose | 0.75% |
| Titanium dioxide | 0.20% |
| FD&C Green #3 | 0.10% |
| Total | 100% |

The inventive methods for making and/or using the hydrogels, and the hydrogels themselves have been described above in considerable detail. This was done for illustrative purposes. Neither the specific implementations of the invention as a whole, nor those of its features, limit the general principles underlying the invention. In particular, the invention is not necessarily limited to the specific constituent materials and proportions of constituent materials used in making the compositions. The invention is also not necessarily limited to hydrogels as specifically composed herein, but extends to other hydrogel applications as well. The specific features described herein may be used in some implementations, but not in others, without departure from the spirit and scope of the invention as set forth. Many additional modifications are intended in the foregoing disclosure, and it will be appreciated by those of ordinary skill in the art that, in some instances, some features of the invention will be employed in the absence of other features. Additional features may be implemented as well. The illustrative examples therefore do not define the metes and bounds of the invention and the legal protection afforded the invention, which function is served by the claims and their equivalents.

Accordingly, what is claimed is:

1. An oral and dental cohesive hydrogel sheet made by:
mixing a hydrogel-forming polymer with water; and
applying energy from an energy source to cross-link the cohesive gel polymer with the water to form a cohesive hydrogel product;
and wherein the energy applied affects the cohesion tackiness;
and further wherein the hydrogel product is substantially free of any additive for enhancing cross-linking.

2. A dental and oral cohesive product according to claim 1 in which the hydrogel sheet is hydrophilic and the hydrogel-forming polymer is a hydrophilic polymer.

3. A dental and oral cohesive product according to claim 2 wherein the hydrophilic polymer is selected from starch, cellulose, cellulose derivatives, polyvinyl alcohol, polyalkylene oxide, polyethylene oxide, polypropylene glycol, poly(1,3-dioxolane), copolymers of polyethylene oxide, copolymers of poly(1,3-dioxolane), polyvinyl pyrrolidone, polyethylene glycol, polyacrylic acid, polymethylene oxide, or a combination thereof.

4. A dental and oral cohesive product according to claim 1, wherein the hydrogel-forming polymer is added to the water in a weight ratio ranging from about 1 part hydrogel-forming polymer to about 33 parts water to about 1 part hydrogel-forming polymer to about 3 parts water.

5. A dental and oral cohesive product according to claim 1, wherein an anti-microbial agent is added and is selected from silver, silver coated fibers, chlorhexidine gluconate, cetylpridium chloride, zinc chloride, copper sulfate, stannous fluoride, triclosan, domaphen bromide, hydrogen peroxide, carbamide peroxide, ethanol, eugenol or a combination thereof.

6. A dental and oral cohesive product according to claim 1, wherein an anti-microbial agent is added and wherein the anti-microbial agent is added to the cohesive hydrogel product in an amount of from about 0.1 to about 3% by weight.

7. A dental and oral cohesive product according to claim 1, wherein an anti-microbial agent is added and wherein the anti-microbial agent or other orally active agent is mixed with the hydrogel-forming polymer and the water prior to application of the energy from the energy source.

8. A dental and oral cohesive product according to claim 1, wherein an anti-microbial agent is added and wherein the anti-microbial orally active agent includes one or more of: a commercially available mouth rinse formulation, benzocaine, sodium fluoride, sodium monofluorophosphate, potassium nitrate, strontium chloride, an artificial flavoring, a natural flavoring, an artificial dye, a natural dye, phenol, cortisone, a steroid, amlexanox, an adhesive gum such as xanthan gum, methyl salicilate, mica, vitamin E, vitamin C, coenzyme Q-10, aloe vera extract, baking soda, chamomile, sodium pyrophosphate, potassium pyrophosphate, sodium benzoate, xylitol, an antifungal preparation, an antiviral preparation, a tartar control agent, amorphous calcium phosphate, crystalline calcium phosphate, calcium, and a peroxygen for the purpose of whitening teeth, or any combination thereof.

9. A dental and oral cohesive product according to claim 8, wherein the orally active substrate is selected and added to a top sheet, a bottom sheet, a scrim, or a combination thereof, and wherein the scrim is selected from a mesh, a foam, a film, a woven material, a non-woven material, or a combination thereof.

10. A dental and oral cohesive product according to claim 1, further comprising an additive selected from a salt, a preservative, a pH adjuster, a cross-linking inhibitor, or a combination thereof.

11. A dental and oral cohesive product according to claim 1, wherein the energy from energy source is selected from an electron beam, gamma radiation, or a combination thereof.

12. A dental and oral cohesive product according to claim 1, wherein the energy from energy source comprises a linear accelerator electron beam.

13. A dental and oral cohesive product according to claim 1 in which an outer coating adhesive is present on the surface of the product to increase the fixative ability of the product.

14. A dental and oral cohesive product according to claim 13 in which the outer coating adhesive is a dry water-activated adhesive such as sodium alginate, xanthan gum, carboxymethylcellulose, carboxyethylccllulose, cocoa butter, glycerine, polyethylene glycol, carbomer, carbopol, starch of any kind or any combination thereof.

15. A hydrogel loaded with an oral care agent for use in treating an oral condition within the oral cavity; whereby the hydrogel is ion beam cross-linked, the hydrogel is adapted to be disposed in the oral cavity for an operative period of time and the oral care agent is controlled-released to treat the oral condition.

16. A hydrogel according to claim 15 wherein the oral condition is alveolitis.

17. A hydrogel material for controlled release of an active substance.

18. A hydrogel according to claim 17 wherein the active substance is one or more of a smoking cessation agent, an appetite suppression agent, and a malodor treatment agent.

19. A hydrogel according to claim 17 loaded with an oral care agent for use in treating an oral condition within the oral cavity; whereby the hydrogel is ion beam cross-linked, the hydrogel is adapted to be disposed in the oral cavity for an operative period of time and the oral care agent is slow-released to treat the oral condition.

20. A hydrogel according to claim 17 loaded with a whitening agent for use in whitening one or more teeth; whereby the hydrogel is ion beam cross-linked, the hydrogel is adapted to be disposed in the oral cavity on or adjacent one or more teeth and the whitening agent is slow-released to whiten the one or more teeth.
